(19) United States
(12) Patent Application Publication
(10) Pub. No.: US 2022/0249299 A1
(43) Pub. Date: Aug. 11, 2022

(54) Child and adult leak-proof garments made with an inner absorbent fabric

(71) Applicants: Shmuel Einhorn, Monroe, NY (US); Shimon Schwartz, Highland Mills, NY (US)

(72) Inventors: Shmuel Einhorn, Monroe, NY (US); Shimon Schwartz, Highland Mills, NY (US)

(21) Appl. No.: 17/586,367

(22) Filed: Jan. 27, 2022

Related U.S. Application Data
(60) Provisional application No. 63/147,746, filed on Feb. 9, 2021.

(51) Int. Cl. A61F 13/49; A41D 31/12

(52) U.S. Cl. CPC A61F 13/49004; A41D 31/125; A61F 13/49006

(57) ABSTRACT

The novel dual-layer garment includes an outer layer made from a fluid-repellant or leak-proof fabric and an inner absorbent layer assembled therewith. The inner layer is made to slow down fluid absorption as compared to regular baby diapers in order to keep the child feeling wet for an extended, yet limited period of time. The adult version of the garment may feature several tight bands along the full length of the garment so as to compartmentalize several sections thereof and limit fluid runoff to avoid an outside leakage of fluids.

### CROSS-REFERENCE DATA

This patent application claims a priority date benefit from the U.S. Provisional Patent Application No. 63/147,746 filed 9 Feb. 2021 and entitled “Potty-training Garment System and Method of Use”, incorporated herein in its entirety by reference.

### BACKGROUND

Without limiting the scope of the invention, its background is described in connection with various garments made using absorbent fabrics. More particularly, the invention describes child and adult outer garments made in part or entirely using a two-layer fabric featuring an inside absorbent fabric layer and an outside non-absorbent fabric layer.

The invention more specifically relates to a child potty-training garment system that absorbs fluid discharge and also conveys a feeling of wetness to the wearer to motivate them to use a toilet. Common children's clothing systems include pants, dresses, blouses, shirts, shorts, skirts, and the like to cover the body of the wear at least in part. For example, a skirt is worn around the waist and covers the legs. One of the problems associated with common children's clothing systems is their limited efficiency. For example, young children are not able to control their bladders and other bodily functions. Bedwetting is a common problem among young children. They must be taught this control. When fluid or other mater is discharged, their clothing (and frequently the bedding sheets) must be changed and cleaned.

Common potty-training solutions to the uncontrolled release of bodily fluids, such as bedwetting, include the use of special absorbent devices such as diapers that quickly capture any fluid and lock it away from the wearer. This, however, teaches the wearer that their lack of control is acceptable as the problem is quickly solved for them. While developments have been made in the area of common clothing systems and potty-training methods, the need exists for further improvements.

The need also exists for fluid-absorbent clothing systems for adults, especially older adults, as well as certain additional groups of users: disabled people (both young and old), patients after certain medical procedures, people requiring nursing assistance, menstruating women, especially with heavy bleeding conditions, and others. Urine and fecal incontinence are known problems with the elderly. Adult diapers are frequently used to address the problem but their fluid holding capacity may not be adequate all the time leaving the user feeling soiled with involuntarily discharged fluids. An overflow of fluids exceeding the holding capacity of an adult diaper causes uncontrolled running of the fluids along the legs of the user and even escaping outside the garment, which may be embarrassing for children as well as the adult user. A need, therefore, exists to prevent this uncontrolled discharge and spare the user from such embarrassments.

Given heightened environmental concerns, the need also exists for reusable fluid-absorbent garments to reduce a large volume of waste associated with discarded disposable diapers after their use.

### SUMMARY

Accordingly, it is an object of the present invention to overcome these and other drawbacks of the prior art by providing a novel dual-layer outer garment configured to absorb unintended leaks for the user.

It is another object of the present invention to provide a novel garment that avoids external leakage of the fluid outside the garment.

It is a further object of the present invention to provide a child potty-training garment that encourages small children to learn proper potty techniques.

The novel dual-layer garment of the invention includes an outer layer made from a fluid-repellant or leak-proof fabric and an inner absorbent layer assembled therewith. The inner layer is made to slow down fluid absorption as compared to regular baby diapers in order to keep the child feeling wet for an extended, yet limited period of time.

The adult version of the garment may feature several tight bands along the full length of the garment so as to compartmentalize several sections thereof and limit fluid runoff to avoid an outside leakage of fluids.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through the use of the accompanying drawings, in which:

FIG. 1 is a front view of a potty-training garment system in accordance with one embodiment of the invention;

FIG. 2 is a front view of an alternative embodiment of the system of FIG. 1;

FIG. 3 is a cross-sectional side view of an alternative embodiment of the system of FIG. 1;

FIG. 4 is a flowchart of a method of teaching bladder control, and

FIG. 5 is a front view of the garment for adult and child users.

### DETAILED DESCRIPTION OF PREFERRED EMBODIMENTS OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

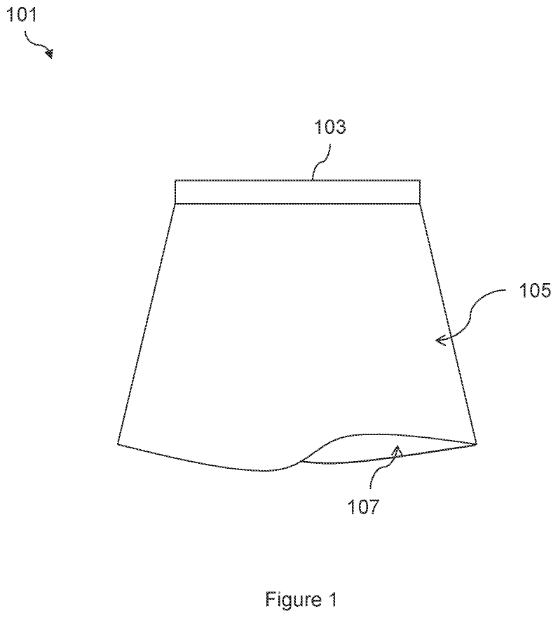

FIGS. 1 through 4 describe the child garment according to various embodiments of the present invention. FIG. 1 depicts a front view of the first embodiment for a potty-training outer garment system in its preferred embodiment. The term “outer garment” is used herein to separate the invention from various diapers and other underwear clothing systems as it can be worn on top or in addition thereto if desired. It will be understood that system 101 provides improvements over common clothing systems and potty-training methods.

The embodiment the system 101 includes a waistband 103 with an outer layer 105 attached thereto and configured to cover the lower abdomen, groin, and a portion of the legs of a person. An inner layer 107 may be attached to the outer layer 105 to form the combined fabric of the garment, which may extend over a part or over the whole garment 101. The waistband 103 and outer layer 105 form a skirt 101, which is shown only as one example of a suitable garment of the invention.

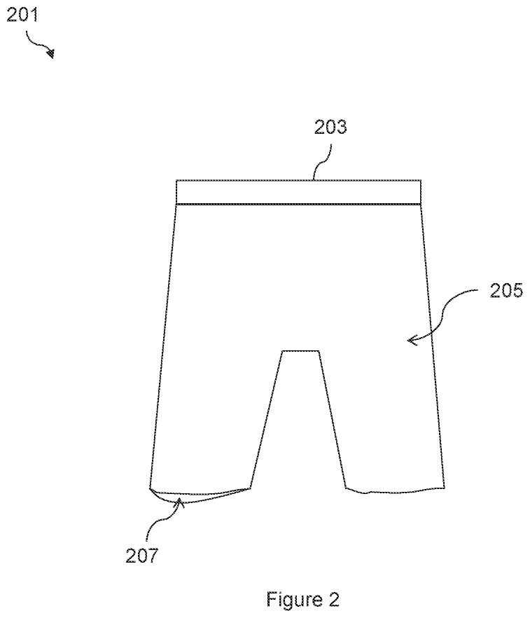

It is contemplated that any type of garment that covers the groin area of the wearer could be used with this invention. An alternative embodiment is provided in FIG. 2. Embodiment 201 includes a waistband 203 that is attached to an outer layer 205. Outer layer 205 may be configured to form two leg holes so that it covers the lower abdomen, groin, and a portion of the legs of a person. The outer layer 205 may have an inner layer 207 attached thereto or integrally formed therewith.

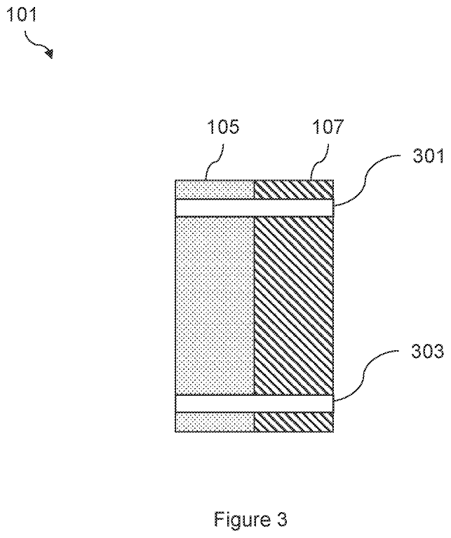

Referring now to FIG. 3, system 101 is depicted in further detail. It is contemplated that the outer layer 105 may be attached to the inner layer 107 at a first location 301 and at least a second location 303. It is contemplated that the outer layer 105 may be configured to act and function as a normal or common garment. In further embodiments, the outer layer 105 may be water-repellent or totally impermeable to fluids. Making the outer layer leak-free with the inner layer being fluid-absorbent creates a complete solution for managing unintended leaks for the user. In further embodiments, the outer layer may be configured to have color patterns and “feel” to touch like regular fabric so as to conceal the fluid-retention capability of the garment and make it appear as a regular piece of clothing.

The inner layer 107 may be configured to partially or slowly absorb fluid. The partial absorption or slow absorption indicates to the wearer that fluid is present in the garment and results in an uncomfortable sensation.

The inner layer 107 may be made using at least one or a combination of bamboo, cotton, wool, fleece, microfiber, hemp, terry, linen, flannel, or other materials.

Various methods of detachable or permanent attachment of the outer layer 105 to the inner layer 107 may be used for the purposes of this invention. For example, a plurality of snaps, buttons, or other spot fasteners may be used throughout the garment to removably or permanently secure both layers of the fabric together. Zippers, line stitching, or similar fastening methods may also be used. In further embodiments, garment seams may be constructed to be leak-proof using impermeable tapes, attached to the inner and outer layers using adhesives, heat-sealing or heat-fusion techniques. Other attachment methods suitable for this invention may include gluing, welding, heat bonding, or fusing two layers together in at least some spots, around the periphery, or over the entire surface of the garment, as the invention is not limited in this regard.

In further embodiments, the inner layer 107 may not be present along the entire inner surface of the outer layer 105. In this case, partial patches of the absorbent inner layer may be provided inside the garment—without covering the entire surface thereof. Each patch may be individually attached to the continuous outer layer 105 using any of the above-mentioned fastening methods.

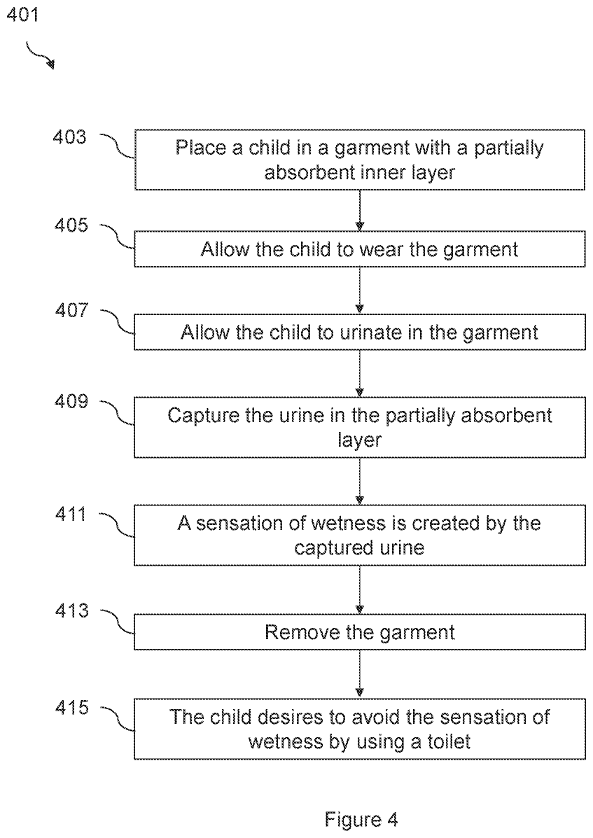

One method to teach potty training according to the invention is depicted in FIG. 4. Method 401 may include placing a child in a garment with a partially absorbent inner layer 403, allowing the child to wear the garment 405, allowing the child to urinate in the garment 407, the partially absorbent layer capturing the urine 409, the captured urine causing a sensation of wetness to the child 411, removing the garment 413, and the child desiring to avoid the sensation of wetness by using a toilet 415.

To achieve the desired effect, the garment may be configured to hold less fluid than a conventional diaper—with the purpose of providing a more acute sense of wetness for the child which would encourage the child to practice proper potty techniques. This may be accomplished by selecting a suitably lower thickness of the inner layer 107.

In other embodiments, the inner layer may be configured to absorb the fluid more slowly than a conventional diaper. In this case, the feeling of wetness would persist only temporarily and for a limited, predetermined period of time. Once the fluid is absorbed fully, the child will not feel wet all the time, which may make the child uncomfortable. The same feature may be helpful to reduce the rate of diaper rash associated with using the garment of the invention.

In further yet embodiments, a thinner patch or a slower absorbency patch of a first inner layer may cover only a portion of the inner surface of the garment in one or more patches, while other portions of the garment may be made fully and quickly absorbent using a second inner layer with a second and higher absorbency rate. In this case, an unpleasant feeling of wetness may be limited to the size and location of that lower absorbency patch, not the entire surface of the garment. In one example, one or two strips of such lower absorbency inner layer material may be positioned on one or both sides of the garment and a fully absorbent inner layer may be positioned in the front and in the back thereof, so as to prevent diaper rash in these more likely locations. In some embodiments, there is no overlap between the first and the second inner layer, while in other embodiments these inner layers may overlap, at least in some places along the garment.

Once the garment is full, it can be machine washed and dried to restore it to the original dry condition and reuse at a later time.

Modern disposable baby diapers include a layer of fluff and a layer of superabsorbent polymer as described in detail by Bachra et al. incorporated herein by reference in its entirety (Yahya Bachra, Ayoub Grouli, Fouad Damiri, Ahmed Bennamara, and Mohammed Berrada. A new approach for assessing the absorption of disposable baby diapers and superabsorbent polymers: A comparative study. Results in Materials, 8:100156, 2020).

FIG. 6 of Bachra is of particular interest. It describes the total tap water absorption capacity of 8 widely marketed diapers as being between 20 and 30 times the dry weight of the diaper. In comparison, a total tap water absorbency capacity for the garment of the present invention may be selected to be lower, such as 5 to 15 times the weight of the garment so as to maintain some sense of the child as being wet. In various embodiments, the total tap water absorbency for the garment of the invention may be selected to be 5, 6, 7, 8, 9, 10, 11, 12, 13, 14, or 15 times the dry weight of the garment. This may be adjusted by varying the thickness of the absorbent inner layer or other characteristics of the inner layer that impact the total absorbency of the fabric.

Another interesting finding in Bachra is seen in FIG. 12 illustrating the absorption kinetics of the diapers. For most diapers, the fluid is mostly absorbed to the diaper's maximum capacity in the first 2-3 minutes of fluid contact. The present invention may use slower absorption fabrics, such as with the total absorption time to saturation being 4-15 min. in various embodiments, the total absorption time to saturation may be selected to be 4 min, 5 min, 6 min, 7 min, 8 min, 9 min, 10 min, 11 min, 12 min, 13 min, 14 min, 15 min or even longer so as to facilitate a prolonged sensation of being wet for the child wearing the garment, thereby urging the child to learn proper potty techniques. This parameter may be adjusted by selecting a suitable extent of fluffiness of the inner layer or by other fabric characteristics that are familiar to those skilled in the art.

Advantageously, one or more tight bands, similar to what is described below, may be included in the design of a child garment, designed to collect the fabric tight around the user's skin, such as with elastic or a draw-string design. In one example, the garment may feature an upper tight band 103 and one or more lower tight bands such as located on the bottom of the garment (not shown). This arrangement may be useful to isolate all leaked fluid and prevent it from leaking outside while the inner layer is slowly absorbing it inside thereof. The feeling of wetness would persist for a longer period of time as compared to conventional diapers, such as at least 5 min—but will still subside soon thereafter. This will encourage the child to learn proper potty techniques, and at the same time avoid the child feeling uncomfortable for a very long period of time.

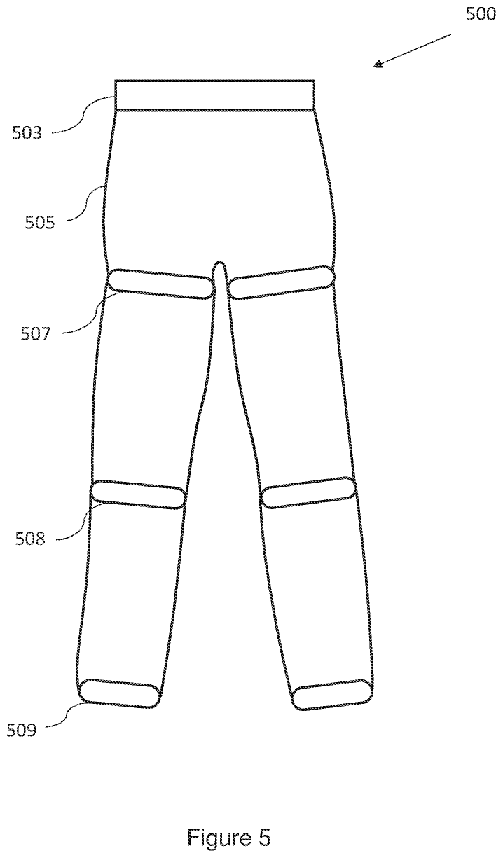

The present invention may also be useful for adult customers as well as children. In particular, FIG. 5 shows full-length pants 500 made from the dual-layer fabric of the invention. Importantly, the term “dual-layer” describes an inner layer and an outer layer, with the inner layer that can be made using one or more individual layers of fabrics as described above. Each individual layer of fabric used for the inner layer of the garment may have a uniform or variable thickness along the length of the garment, for example, with greater thickness around the groin area facilitating greater fluid retention.

The top waistband portion 503 may be an elastic or a draw-string component that allows the user to tighten the garment around their waist. Advantageously, the garment may have one or more additional elastic or draw-string bands 507, 508, 509, where the fabric of the garment may be closely collected around the limb of the user, so as to prevent fluid runoff down the limb, a common problem for incontinent adults.

As described in FIG. 7 by Bachra, fluid runoff can occur with any diaper, both child and adult diapers, which may occur prior to as well as after diaper saturation. Once a conventional diaper is full, the fluid has no more room to be absorbed. Using an extended length of the garment as seen in FIG. 5 allows additional absorbent material to be located along the upper and lower portions of the limb so as to provide additional fluid absorption capability.

In addition, one or several areas where the fabric may be tightly collected around the limb of the user. This may assist to avoid leaking of the fluid outside the garment, thereby avoiding embarrassment for the user, both children and adults. FIG. 5 shows examples of specific contemplated locations of the tight bands collecting the fabric closely around the limb, such as middle tight bands 507 at the bottom of the trunks, alternative middle tight bands 508 around the knees, and lower tight bands 509 at the area of the user's ankles. Other or additional tight fabric bands may be also provided in other locations as the invention is not limited in this regard.

Although FIG. 5 shows an example of the pants, the invention is not limited to that type of garment. Skirts, overalls, pajama pieces or full sets, or other lower and upper body garments may also be made using the dual-layer fabric of the invention.

Furthermore, another advantageous feature of the garment is the ability to remove it without the need to take down the garment like regular pants. To facilitate gentle sideways removal, side snaps, buttons, zippers, Velcro, or other similar fasteners may be provided along the entire length of one or both sides of the garment (not shown in the drawings). Undoing these fasteners along the sides of the garment may help to facilitate careful sideways removal of the soiled garment without causing additional fluid leaks during the process of garment exchange.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Incorporation by reference is limited such that no subject matter is incorporated that is contrary to the explicit disclosure herein, no claims included in the documents are incorporated by reference herein, and any definitions provided in the documents are not incorporated by reference herein unless expressly included herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step, or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of A, B, C, Aft AC, BC, or ABC, and if the order is important in a particular context, also BA, CA, CB, CBA, BCA, ACB, BAC, or CAB. Continuing with this example, expressly included are combinations that contain repeats of one or more items or terms, such as BB, AAA, Aft BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit, and scope of the invention. All such similar substitutes and modifications apparent to those skilled in the art are deemed to be within the spirit, scope, and concept of the invention as defined by the appended claims.

What is claimed is:

1. A dual-layer reusable absorbent outer garment comprising a leak-proof outer layer and a fluid-absorbent inner layer assembled therewith, the inner layer is made from at least one fabric selected from a group consisting of bamboo, cotton, wool, fleece, microfiber, hemp, terry, linen, and flannel, wherein the inner layer is configured to extend fluid absorption time to at least 5 min, the garment further comprising an upper tight band and at least one lower tight bands characterized in that at each tight band the inner layer is collected tightly around user's skin to prevent fluid leakage across thereof, thereby the fluid is absorbed at a reduced rate while still contained within the garment and prevented from leaking outside thereof.

2. The dual-layer garment as in claim 1, wherein the garment further comprises one or more fasteners on one side thereof or on both sides thereof, whereby removal of a soiled garment may be accomplished after opening of all fasteners without a risk of spilling the fluid.

3. The dual-layer outer garment as in claim 1, wherein the inner layer is present in one or more patches only along a portion of the garment as defined by the outer layer.

4. The dual-layer garment as in claim 1, wherein one or more patches of a first inner layer with a first absorbance capacity is present at some portions of the garment along the outer layer, and one or more patches of a second inner layer with a second fluid absorbance capacity is present along other portions of the garment without an overlap between the first inner layer and the second inner layer.

5. The dual-layer garment as in claim 4, wherein the second fluid absorbance capacity is higher than the first fluid absorbance capacity.

6. The dual-layer garment as in claim 5, wherein the first inner layer is located along the sides of the garment and the second inner layer is located along the front and back of the garment.

7. A dual-layer reusable absorbent outer garment comprising a leak-proof outer layer and a fluid-absorbent inner layer assembled therewith, the inner layer is made from at least one fabric selected from a group consisting of bamboo, cotton, wool, fleece, microfiber, hemp, terry, linen, and flannel, the garment further comprising an upper tight band and at least one middle tight band characterized in that at each tight band the inner layer is collected tightly around subject's skin to prevent fluid leakage across thereof, thereby the fluid is absorbed while still contained within the garment and prevented from leaking outside thereof.

8. The dual-layer garment as in claim 7 further comprising one or more lower tight bands at the bottom of the garment for further preventing fluid from leaking outside thereof.

9. The dual-layer garment as in claim 7, wherein the tight band is made as elastic or draw-string band configured to collect garment fabric tightly around user's skin to prevent fluid leakage across thereof.
